(19) United States
(12) Patent Application Publication
(10) Pub. No.: US 2023/0250411 A1
(43) Pub. Date: Aug. 10, 2023

(54) CELLULAR REPROGRAMMING OF HUMAN ADIPOCYTES

(71) Applicant: MIAMI UNIVERSITY, OXFORD, OH (US)

(72) Inventors: PAUL A. HARDING, OXFORD, OH (US); KAYLA CARTWRIGHT, ELMHURST, IL (US); SEAN R. TAYLOR, CINCINNATI, OH (US); ABBY SFERRELLA, INDIANAPOLIS, IN (US)

(21) Appl. No.: 18/161,467

(22) Filed: Jan. 30, 2023

Related U.S. Application Data
(60) Provisional application No. 63/305,299, filed on Feb. 1, 2022.

(51) Int. Cl. C12N 9/64; C12N 15/86; C12N 5/077; A61K 35/761

(52) U.S. Cl. CPC C12N 9/6416; C12N 15/86; C12N 5/0653; A61K 35/761; C12N 2710/10041

(57) ABSTRACT

Obesity and related conditions and diseases pose ever increasing issues for the developed world. There is a need for interventions that can treat/prevent the underlying causes of e.g., diabetes and obesity. Accordingly compositions and methods for altering at least one expression level related to cellular metabolism are disclosed herein.

### CROSS-REFERENCE TO RELATED APPLICATIONS

This application claims the benefit of U.S. Provisional Application No. 63/305,299 filed on Fe. 1, 2022, the disclosure of which is hereby incorporated by reference in its entirety.

### FIELD

The present disclosure relates to compositions for and methods of reprogramming cells. More particularly, to compositions for and methods of manipulating the expression levels of a cell to increase one or more markers associated with Brown Adipocyte Tissue.

### BACKGROUND

Obesity is one of the most burdensome health issues today for developed nations, both in terms of life expectancy and overall well-being of the population. Lifestyle choices combined with calorie-dense food contribute to the dramatic increases in the disease seen in both children and adults. The convenience of fast-food and the ease of socializing through a phone has created a sedentary culture leading to poor lifestyle choices and an increase in obesity.

An individual is considered obese by having a BMI≥30 kg/m². The current increase in the prevalence of obesity in the first world is of concern because of the vast number of diseases associated with it. These include type II diabetes, hypertension, metabolic syndrome, and many types of cancer. In general, obesity has been connected to an increased risk of morbidity, mortality, and reduced life expectancy. As a result, the healthcare costs linked to obesity have increased and are expected to continue to rise in much of the developed world. There is an urgent need for novel interventions to reduce individual weight and prevent obesity related metabolic diseases.

Typically, adipose tissue is best known for its role in fat storage, but specialized heat-producing adipocytes can also suppress weight gain and metabolic disease. Brown adipocytes (or brown adipose tissue: BAT) are a key site of this heat production. In obligate hibernators, BAT thermogenesis is essential for keeping animals warm during their hibernation periods. These BAT cells contain increased numbers of mitochondria and uncoupling protein-1 (UCP-1) which short circuits the electrochemical gradient that drives ATP synthesis. When the organism is activated by a stimulus, such as the cold or glucocorticoids, the increase of respiratory chain function causes the combustion of available substrates producing heat which is then distributed throughout the body. These substrates oftentimes include glucose molecules and stored lipids. Thus, their combustion depletes the body's energy stores and decreases overall weight.

### SUMMARY

The general inventive concepts are based, in part, on the recognition that cells, including but not limited to differentiated cells, can be reprogrammed or transdifferentiated into brown adipose tissue (BAT). It has surprisingly been found that co-expression of heparin-binding EGF-like growth factor (HB-EGF) and A Disintegrin and Metalloproteinase (ADAM) 12S can reprogram cells into BAT-like cells. The BAT-like reprogrammed cells phenotypically and physiologically appear similar to BAT cells in that they exhibit at least one of the following properties (markers of BAT): (small) lipid droplet accumulation, increase in the number of mitochondria (those of skill in the art will recognize the relative lack of mitochondria in white adipose tissue (WAT)), cellular quiescence, increased BAT gene expression (PRDM16, PGC-1a, UCP-1), decreased WAT gene expression (PPARg, C/EBPa, AKT-1), increased gene expression in cellular reprogramming related genes (FGF-2, KLF3/4, HOXA10, and HOXC5), decreased gene expression in a cellular differentiation gene, LMNA, increased oxygen consumption and extracellular acidification rates compared to control cells (i.e., increased glycolysis).

The general inventive concepts are based on the surprising discovery that Applicants demonstrated cellular reprogramming in multiple cell types including: mouse fibroblasts, human A431 epidermoid carcinoma cells, human embryonic kidney (HEK) 293 cells, and human primary adipocytes collected from liposuction cells.

In certain exemplary aspects, the general inventive concepts contemplate a method of enhancing at least one BAT-like expression level in an individual. The method comprises identifying an individual in need of increased expression of at least one BAT-like marker; providing an expression vector comprising a nucleotide sequence encoding ADAM 12S (as used herein, unless otherwise noted, ADAM 12S refers to ADAM 12S or substantially similar sequences encoding ADAM 12S or active fragments thereof that will express an ADAM 12 polypeptide protease when introduced into the nucleus of an animal cell)operatively linked to a promoter; and contacting at least one cell of the individual with the expression vector.

In certain exemplary aspects, the general inventive concepts contemplate a method of treating a condition associated with at least one of obesity, metabolic syndrome, and insulin resistance by reprogramming at least one differentiated cell in an individual. The method comprises identifying an individual diagnosed with at least one of obesity, metabolic syndrome, and insulin resistance; providing an expression vector comprising a nucleotide sequence encoding ADAM 12S operatively linked to a promoter; and contacting at least one cell of the individual with the expression vector.

In certain exemplary aspects, the general inventive concepts contemplate a composition for altering at least one BAT expression level in a cell, the composition comprising an expression vector comprising a nucleotide sequence encoding ADAM 12S operatively linked to a promoter, wherein the composition does not comprise Ad-FIB-EGF.

In certain exemplary aspects, the general inventive concepts contemplate a method of treating or preventing diabetes in an individual. The method comprising reprograming at least one cell to a BAT cell according to the methods and compositions discussed herein.

In certain exemplary aspects, the general inventive concepts contemplate a method of treating or preventing obesity in an individual. The method comprising reprograming at least one cell to a BAT cell according to the methods and compositions discussed herein.

In certain exemplary aspects, the general inventive concepts contemplate a method of lowering measured glucose levels in an individual. The method comprising reprograming at least one cell to a BAT cell according to the methods and compositions discussed herein.

In certain exemplary aspects, the general inventive concepts contemplate a method of reprogramming a primary adipocyte. The method comprising contacting a human primary adipocyte with ADAM 12S or a vector (e.g., virus, plasmid) comprising ADAM 12S, thereby stimulating cellular reprogramming of the cell into BAT-like cells (i.e., the cell exhibits at least one of the aforementioned properties/markers of BAT) according to the methods and compositions discussed herein.

Other aspects and features of the general inventive concepts will become more readily apparent to those of ordinary skill in the art upon review of the following description of various exemplary embodiments in conjunction with the accompanying figures.

### BRIEF DESCRIPTION OF THE DRAWINGS

The general inventive concepts, as well as embodiments and advantages thereof, are described below in greater detail, by way of example, with reference to the drawings in which:

FIG. 1 is a schematic of Adenoviral Vectors. pShuttle 1 and 2 and pAdEasy-1 adenoviral backbone vectors. HB-EGF was cloned into pShuttle-IRES-hrGFP-2 while ADAM 12S was cloned into pShuttle-IRES-hrGFP-1. Which were used to transfer the gene of interests into pAdEasy-1 adenoviral backbone vector. These images were modified from Agilent Technologies and www.the-scientist.com.

FIG. 2 shows RT-PCR Detection of HB-EGF in Human Primary Preadipocytes. Total cellular RNA was isolated from human primary adipocytes to identify the presence of HB-EGF mRNA. A human HB-EGF forward primer (+; 5′-ATGAAGCTGCTGCCGTCGG-3′) and reverse primer (−; AGTGGGAATTAGTCATGCCC-3′) resulted in a 627bp HB-EGF PCR product in the presence of reverse transcriptase (+RT) and no detectable PCR product in the absence of reverse transcriptase (−RT), as shown above.

FIG. 3 shows Lipid Accumulation in Ad-ADAM 12S Infected Human Primary Preadipocytes. Human Primary Preadipocytes obtained from ZenBio were cultured in a 6-well plate and infected with 15 μl of Ad-ADAM 12S. After 48 hours, fluorescent microscopy was conducted and GFP was observed (B,D) indicating infection of the cells by the adenovirus. Brightfield images of the primary preadipocytes that have been infected with Ad-ADAM 12S show increased lipid accumulation (A,C). Images were taken using the 20× objective lens (Olympus).

FIG. 4 shows a lack of Lipid Accumulation in Ad-MOCK Infected Human Primary Preadipocytes. Human Primary Preadipocytes obtained from ZenBio were cultured in a 6-well plate and infected with 15 μl of Ad-MOCK. Fluorescent microscopy conducted 48 hours after infection indicate a presence of GFP (B,D), demonstrating that the cells were infected by the adenovirus. Brightfield images of the human primary preadipocytes indicated that while the cells were infected with the adenovirus, there is no noticeable lipid accumulation (A,C). Images were taken using the 20× objective lens (Olympus).

FIG. 5 is an image showing Oil Red O Staining in Human Primary Preadipocytes. Human Primary Preadipocytes infected with either Ad-ADAM 12S (A,B) or Ad-MOCK (C,D) were fixed and stained with Oil Red O and then counterstained with hematoxylin. Ad-ADAM 12S infected cells display a tremendous amount of Oil Red O staining compared to Ad-MOCK infected cells. Images were taken using the 20× objective lens (Olympus).

FIG. 6 is a graph of Metabolic output of Human Primary Preadipocytes. Human Primary Adipocytes were infected with either Ad-ADAM 12S (n=10) or Ad-Mock (n=10). Metabolic parameters were measured using Seahorse XF24 at basal, after catecholamine exposure, and when stressed with a mix of FCCP+Oligomycin exposure. Extracellular Acidification Rate (ECAR) was measured to determine the rate of glycolysis in the human primary adipocytes. * compares the basal rate of ADAM 12S infected cells to the stressed rate. ** compares ADAM 12S to MOCK cells for each treatment type, either stressor mix or catecholamine exposure. (p-value<0.05).

### DETAILED DESCRIPTION

Several illustrative embodiments will be described in detail with the understanding that the present disclosure merely exemplifies the general inventive concepts. Embodiments encompassing the general inventive concepts may take various forms and the general inventive concepts are not intended to be limited to the specific embodiments described herein.

While various exemplary embodiments are described or suggested herein, other exemplary embodiments utilizing a variety of methods and materials similar or equivalent to those described or suggested herein are encompassed by the general inventive concepts.

Unless defined otherwise, all technical and scientific terms used herein have the same meaning as commonly understood by one of ordinary skill in the art to which the invention belongs.

Adipose tissue as used herein refers to tissue composed primarily of adipocytes (i.e., fat cells), and functions primarily to store energy in the form of fat. Adipose tissue is characterized as either white adipose tissue (WAT) or brown adipose tissue (BAT).

White adipose tissue is composed primarily of white fat cells that contain a large lipid droplet surrounded by a layer of cytoplasm. The nucleus in white fat cells is flattened and located on the periphery. A typical white fat cell is about 0.1 mm in diameter, with some being twice that size and others half that size. The fat within a white fat cell is stored in a semi-liquid state, and is composed primarily of triglycerides and cholesteryl ester.

Brown adipose tissue is composed primarily of brown fat cells. Unlike white fat cells, brown fat cells have considerable cytoplasm, with lipid droplets scattered throughout. The nucleus is round and is not located in the periphery of the cell. Brown fat cells also include a large quantity of mitochondria, which result in the brown color of the cells. Brown adipose tissue is also known as “baby fat” and is used physiologically to generate heat. Brown adipose tissue has a different embryological origin than white adipose tissue, and appears to share the same lineage as muscle. Enerback S, N Engl J Med 360, p. 2021-2023 (2009).

The term “BAT-like cell” as used herein, refers to cell that demonstrates at least one marker of BAT, including, but not limited to: lipid droplet accumulation, increased number of mitochondria, cellular quiescence, increased BAT gene expression (e.g., PRDM16, PGC-1a, UCP-1), decreased white adipose tissue (WAT) gene expression (e.g., PPARg, C/EBPa, AKT-1), increased gene expression in cellular reprogramming related genes (FGF-2, KLF3/4, HOXA10, and HOXC5), decreased gene expression in a cellular differentiation gene, LMNA, increased oxygen consumption and extracellular acidification rates compared to control cells (i.e., increased glycolysis).

The term “vector” refers to a nucleic acid capable of transporting another nucleic acid to which it has been linked. One type of vector that can be used in accord with the presently disclosed subject matter is a retroviral vector, i.e., a nucleic acid capable of integrating the nucleic acid sequence of interest into the host cell chromosome. Other vectors include those capable of autonomous replication and expression of nucleic acids to which they are linked. Vectors capable of directing the expression of genes to which they are operatively linked are referred to herein as “expression vectors”. In general, expression vectors of utility in recombinant DNA techniques are often in the form of plasmids. However, the presently disclosed subject matter is intended to include such other forms of expression vectors which serve equivalent functions (e.g., retroviral vectors such as adenoviral vectors, cosmids or bacmids) or and which become known in the art subsequently hereto.

The term “expression vector” as used herein refers to a DNA sequence capable of directing expression of a particular nucleotide sequence in an appropriate host cell. Expression vectors can contain a variety of control sequences (e.g., promoters and terminators), structural genes (e.g., genes of interest), and nucleic acid sequences that serve other functions as well. The nucleotide sequence of interest, including any additional sequences designed to effect proper expression of the nucleotide sequences, can also be referred to as an “expression cassette”.

The term “transfection” refers to the introduction of a nucleic acid, e.g., an expression vector, into a recipient cell (e.g., a primary adipose tissue cell), which in certain instances involves nucleic acid-mediated gene transfer. The term “transformation” refers to a process in which a cell's genotype is changed as a result of the cellular uptake of heterologous nucleic acid. For example, a transformed cell can express a protein such as HB-EGF or ADAM 12 that is not present or not present at elevated levels in the typical genotype of the cell prior to transformation. In some embodiments of the invention, the expression vectors are introduced directly to a subject in vivo.

The term “operatively linked” as used herein refers to bringing a polynucleotide coding sequence under the control of a promoter. Operatively linking a sequence to a promoter means one positions the 5′ end of the transcription initiation site of the transcriptional reading frame of the protein between about 1 and about 50 nucleotides “downstream” of (i.e., 3′ of) the chosen promoter. Appropriate spacing of promoters and the polynucleotide sequences whose expression they govern is well known by those skilled in the art.

The polynucleotide encoding a gene product is under the transcriptional control of a promoter. A “promoter” as used herein refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The terms “susceptible” and “at risk” as used herein, unless otherwise specified, mean having little resistance to a certain condition or disease relative to the general population (e.g., individuals with a family history of obesity, metabolic syndrome, insulin resistance), including being genetically predisposed, having a family history of, and/or having symptoms of the condition or disease. The term refers to those having a vulnerability to conditions such as obesity, metabolic syndrome, and/or insulin resistance that is higher than the general population.

The terms “modulating” or “modulation” or “modulate” as used herein, unless otherwise specified, refer to the targeted movement of a selected characteristic or symptom.

The term “ameliorate” as used herein, unless otherwise specified, means to eliminate, delay, or reduce the prevalence or severity of symptom(s) associated with a condition or disease.

The term “an effective amount” or “therapeutically effective amount” or “amount sufficient to” is intended to qualify the amount of an active ingredient (e.g., pharmaceutical composition or vector) which will achieve the goal of modulating, preventing, ameliorating, or treating a symptom, disease, or condition or that which will achieve the goal of decreasing the risk that the individual will suffer an adverse health event, including reducing or preventing one or more symptoms, while avoiding adverse side effects such as those typically associated with alternative therapies. The effective amount may be administered in one or more doses.

The terms “treating” and “treatment” as used herein, unless otherwise specified, includes delaying the onset of a condition, reducing the severity of symptoms of a condition, or eliminating some or all of the symptoms of a condition including obesity, type II diabetes, metabolic syndrome, insulin resistance, among other related conditions discussed herein.

The transformation of a cell with a heterologous nucleic acid (for example, an expression vector) can be characterized as transient or stable. As used herein, the term “stable” refers to a state of persistence that is of a longer duration than that which would be understood in the art as “transient”. These terms can be used both in the context of the transformation of cells (for example, a stable transformation), or for the expression of a transgene (for example, the stable expression of a gene encoding a sugar metabolizing enzyme) in a transgenic cell. In some embodiments, a stable transformation results in the incorporation of the heterologous nucleic acid molecule (for example, an expression vector) into the genome of the transformed cell. As a result, when the cell divides, the vector DNA is replicated along with the endogenous genome so that progeny cells also contain the heterologous DNA in their genomes. Transformation of plasmid or other self replicating organelle based nucleic acids (e.g., mitochondrial DNA) is also contemplated under this definition.

The term “transdifferentiation” (also referred to as reprogramming) as used herein describes the conversion of a non-stem cell into a different type of cell. Transdifferentiation is a type of metaplasia, which is the conversion of one differentiated cell type into another differentiated cell type. It is generally caused by some sort of abnormal stimulus such as a dramatic change in the cell's environment. Cell types are distinct morphological or functional forms of cells. The term also refers to the targeted modification or modulation of the expression level(s) of a target cell or tissue. In certain aspects, the term refers to modulating (e.g., increasing) at least one marker characteristic of BAT in the cell. In certain aspects, the term refers to modulating at least one marker for metabolic activity in the cell. In certain aspects, the term refers to modulating at least one marker for glycolytic activity.

A variety of different types of cells can be caused to transdifferentiate into brown adipose cells as a result of transfection with an expression vector including the polynucleotide sequences for ADAM 12 (or an active fragment thereof). The transdifferentiated cells can be animal cells or mammal cells.

Transdifferentiation of cells in a tissue may result in varying percentages of conversion to BAT-like cells. The number of cells converted depends on a variety of factors. In some embodiments, up to 100% of the cells in a target tissue can be converted, whereas in other embodiments up to 90%, 80%, 70%, 60%, 50%. 40%, 30%, 20%, 10%, or 5% of the animal/host target cells are converted to BAT-like cells. These brown adipose tissue cells will be characterized by exhibiting at least one marker of BAT as discussed previously.

The general inventive concepts relate to systems for and methods of (re)programming cells. While the discussion presented herein is focused on human and/or mammalian cellular reprogramming, those of ordinary skill in the art will recognize the applicability of the cellular reprogramming described herein is not limited to the specific embodiments discussed herein.

In certain exemplary aspects, the general inventive concepts contemplate a method of treating or preventing diabetes in an individual. The method comprising reprograming at least one cell to a BAT cell according to the methods and compositions discussed herein.

In certain exemplary aspects, the general inventive concepts contemplate a method of treating or preventing obesity in an individual. The method comprising reprograming at least one cell to a BAT cell according to the methods and compositions discussed herein.

In certain exemplary aspects, the general inventive concepts contemplate a method of treating or preventing metabolic syndrome in an individual. The method comprising reprograming at least one cell to a BAT cell according to the methods and compositions discussed herein.

In certain exemplary aspects, the general inventive concepts contemplate a method of lowering measured glucose levels in an individual. The method comprising reprograming at least one cell to a BAT cell according to the methods and compositions discussed herein.

In certain exemplary aspects, the general inventive concepts contemplate a method of reprogramming a primary adipocyte. The method comprising contacting a human primary adipocyte with ADAM 12S, thereby stimulating cellular reprogramming into BAT-like cells. according to the methods and compositions discussed herein.

The general inventive concepts are based, in part on the discovery that cells can be reprogrammed or transdifferentiated into BAT-like cells (i.e., cells that demonstrate an increase in at least one BAT marker) by utilizing endogenous hHB-EGF (rather than introducing HB-EGF into the cell) and contacting the cells (e.g., human primary adipocytes) with ADAM 12S, stimulating cellular reprogramming into BAT-like cells.

While not wishing to be bound by theory, Applicants believe that by reprogramming cells to demonstrate at least one marker of a BAT cell, conditions such as diabetes, obesity, insulin resistance, metabolic syndrome, among others, can be treated. The cells reprogrammed according to the general inventive concepts function as metabolically active BAT cells with an increased glycolytic rate. Increased glycolysis is necessary for BAT as brown adipocytes rely on glucose consumption to achieve maximum thermogenic output. Non-shivering thermogenesis is a highly energetic process and relies on a readily available fuel supply of circulating glucose or free fatty acids from white adipose tissue. In this way, BAT is effective at decreasing blood glucose levels and reducing the amount of white fat that would otherwise be present in the individual, acting as a potential therapeutic target for type II diabetes and obesity, among others. The uptake of glucose and lipid substrates by BAT occurs through upregulation of glucose transporters and lipoprotein lipase. Consequently, BAT activation is able to increase insulin secretion and decrease insulin resistance through the dissipation of excess fuel in the body. This provides a vast opportunity for BAT's use in developing ways to combat the devastating consequences of metabolic disease(s).

The instant examples show that the ADAM 12S modified (transfected) cells demonstrate noticeable lipid droplet accumulation in comparison to the mock infected cells which was confirmed by significant and specific Oil Red O staining. BAT gene PGC-1α was found to be statistically significantly upregulated in the cells that were infected with ADAM 12S (p=0.05), while the mock infected cells did not exhibit this pattern of gene expression. The BAT-like gene UCP-1 was upregulated but not statistically significant (p=0.05). PRDM16, a BAT-like gene, was found to be statistically significantly downregulated in ADAM 12S infected cells. The Seahorse metabolic assay demonstrated an increased rate of glycolysis for cells contacted with ADAM 12S after exposure to a stressor mix, composed of FCCP and Oligomycin, when compared to their basal rate. ECAR was significantly increased in ADAM 12S cells compared to MOCK cells after exposure to catecholamines and the stressor mix (FCCP and Oligomycin). These novel insights provide the first evidence demonstrating BAT-like cellular reprogramming occurs in vivo in humans. The general inventive concepts are applicable to combat obesity, type II diabetes, and other similar conditions and diseases.

The general inventive concepts are based, in part, on the recognition that cells, including but not limited to differentiated cells, can be reprogrammed or transdifferentiated into cells that express at least one marker of BAT. It has surprisingly been found that co-expression of heparin-binding EGF-like growth factor (HB-EGF) and A Disintegrin and Metalloproteinase (ADAM) 12S can reprogram cells into BAT-like cells. The BAT-like reprogrammed cells phenotypically and physiologically appear similar to BAT cells in that they exhibit at least one of the following properties (markers of BAT): lipid droplet accumulation, increase in the number of mitochondria, cellular quiescence, increased BAT gene expression (PRDM16, PGC-1a, UCP-1), decreased white adipose tissue (WAT) gene expression (PPARg, C/EBPa, AKT-1), increased gene expression in cellular reprogramming related genes (FGF-2, KLF3/4, HOXA10, and HOXC5), decreased gene expression in a cellular differentiation gene, LMNA, increased oxygen consumption and extracellular acidification rates compared to control cells (i.e., increased glycolysis).

Obesity and the associated metabolic complications that result have proven to be a significant clinical problem. Traditional lifestyle changes have shown to be ineffective due to lack of adherence to interventions long term. Novel solutions are needed to prevent and correct the obesity epidemic. BAT provides a new solution to increase metabolic rate and counteract obesity and the resulting comorbidities that have become increasingly common today. BAT's role in metabolic rate and glucose and lipid metabolism make it a potential therapeutic target to combat metabolic disease in humans.

BAT's potential as means for weight loss combined with the knowledge that BAT is not just found in babies but is present in adult humans and persists into adulthood, make it a unique mechanism in the struggle against obesity and related diseases. Applicant's previous studies have shown that BAT-like cellular reprogramming occurs when a cell is co-infected with Ad-HB-EGF and Ad-ADAM 12S (see e.g., U.S. Pat. No. 8,455,191, the entire content of which is hereby incorporated by reference as if recited in its entirety herein). The instant Examples are surprising and unexpected inasmuch as they demonstrate that in human preadipocytes endogenous HB-EGF, evident through RT-PCR, can be utilized, thus requiring only ADAM 12S infection in human preadipocytes (i.e., no exogenous HB-EGF is required) to produce cells that exhibit/express at least one marker of BAT. Single infection of preadipocytes by ADAM 12S was the mechanism by which this experiment was conducted and resulted in phenotypically indicative brown adipose tissue. BAT characteristically has multilocular fat droplets and this was observed in our infected cells both under the microscope and when confirmed by significant Oil Red O staining.

After confirming the phenotypically typical BAT-like nature of the cells, further studies were conducted to determine if the BAT-like cells behaved like brown adipose tissue with respect to cellular expression. Differential gene expression showed upregulation of BAT marker genes UCP-1 and PGC-1α. PGC-1α is an essential gene involved in brown fat thermogenesis and many aspects of mitochondria biology. PGC-1α is responsible for mitochondrial biogenesis and function, which explains its role in BAT thermogenesis due to the dissipation of heat during the electron transport chain. This further corresponds with PGC-1α's role in inducing UCP-1 gene expression, which allows for the proton gradient to circumvent ATP synthase resulting in brown fat's thermogenic role. Upregulation of these essential BAT genes confirms that the infected cells are indeed not just phenotypically reminiscent of BAT but have been transformed to function with the same genes known to be associated with and characteristic of brown adipose relative to WAT. One of the BAT genes that was analyzed, PRDM 16, was found to be downregulated. While PGC-1α and UCP-1 are essential for brown fat thermogenesis, they are not involved in brown fat differentiation like PRDM 16. PRDM 16 controls the switch which determines the fate of a cell to be either skeletal myoblasts or brown fat cells. PRDM 16 downregulation is likely due to the timing of the experiment being conducted after the cell was committed to brown fat differentiation.

Studies were conducted to determine the metabolic potential of the BAT-like cells. Seahorse technology utilizes oxygen consumption (OCR) and extracellular acidification rates (ECAR) to determine the metabolic potential of cells. Mitochondrial respiration and glycolysis are measured under baseline and stressed conditions to determine the cells response to increased energy demand, or their metabolic potential. ECAR is a measure of the rate of glycolysis of the cells and was found to be similar for ADAM 12S and MOCK infected human primary preadipocytes at basal conditions. With similar baseline rates, the cells were then stressed with catecholamines to demonstrate the effect of glucocorticoids on BAT. Glucocorticoids are known to stimulate growth, proliferation and thermogenesis in brown adipocytes. As expected, ADAM 12S-contacted cells had a significantly higher ECAR compared to MOCK infected cells. The cells were then further stressed with a stressor compound containing FCCP and Oligomycin compounds. Oligomycin binds to ATP synthase, inhibiting ATP production and causing an increase in the rate of glycolysis as cells are compensating to meet their energy demands. FCCP depolarizes the mitochondrial membrane driving oxidative phosphorylation in an attempt to restore membrane potential. The stressor mix resulted in significantly increased ECAR in ADAM 12S cells. Not only was the ECAR of ADAM 12S cells significantly increased compared to MOCK infected cells, but ECAR or the rate of glycolysis was significantly increased compared to the basal rate of these BAT-like cells. This increased rate of glycolysis demonstrates that ADAM 12S infected cells have increased metabolic potential compared to MOCK infected cells.

Overall, ADAM 12S expression in human primary adipocytes stimulates cellular reprogramming with the use of endogenous hHB-EGF that results in the upregulation of BAT genes UCP-1 and PGC-1α. These BAT-like cells have increased metabolic potential reminiscent of BAT when stressed with catecholamines and a stressor mix of FCCP and Oligomycin. Increased glycolytic rates in these brown adipose cells provides support for the use of BAT as a novel treatment for obesity and type II diabetes, among other conditions and diseases.

The following examples illustrate features and/or advantages of the compositions, systems, and methods according to the general inventive concepts. The examples are given solely for the purpose of illustration and are not to be construed as limitations of the general inventive concepts, as many variations thereof are possible without departing from the spirit and scope of the general inventive concepts.

### Materials and Methods

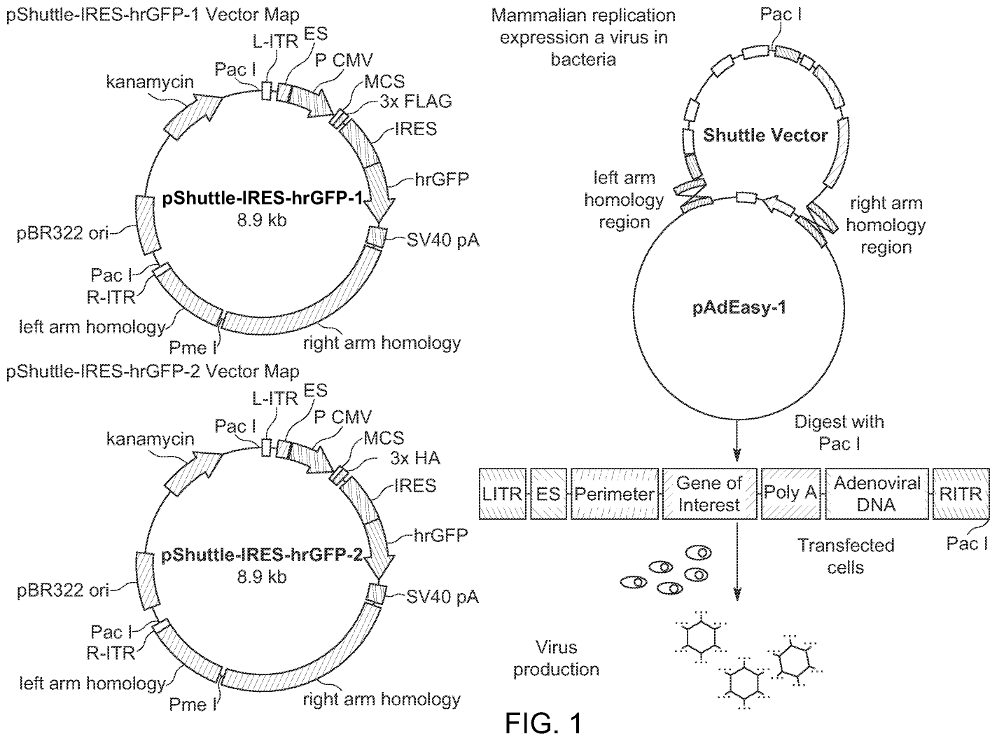

Generation of ADAM 12S adenovirus: AdEasy adenoviral system (Agilent Technologies, Santa Clara, Calif.) was used to create an ADAM 12S adenovirus. The gene of interest (GOI) ADAM 12S cDNA (2.1 kb) was cloned into a plasmid shuttle vector that makes use of an internal ribosomal entry site allowing for the expression of humanized recombinant green fluorescent protein (pShuttle-IRES-hrGFP-1). The sense primer for ADAM 12S contains a Spe1 site, while the anti-sense primer contains a Xho1 site and thus was digested, gel purified, and inserted into pShuttle-IRES-hrGFP-1 that was also digested with Spe1 and Xho1. Additionally, ADAM 12S lacked a stop codon and incorporation of a HA tag for detection. Recombination of shuttle vectors with pAd-Easy vector (FIG. 1), providing the adenoviral backbone, occurred in BJ5183-AD-1 cells. BJ5183-AD-1 cells were transformed by electroporation (200Ω, 2.5 kV, 25 μF) with shuttle vector (50 ng) and pAdEasy-1 supercoiled vector (100 ng) and incubated in S.O.C. media at 37° C. for one hour in a temperature controlled shaker and then plated on kanamycin agar plates. Plates were incubated overnight at 37° C. Recombinant colonies were selected and propagated in LB media at 37° C. in a temperature controlled shaker overnight. DNA was characterized using a mini-prep DNA isolation kit (Qiagen, Venlo, Netherlands) and gel purified in 0.8% agarose TAE gel. The plasmids isolated by gel purification were then digested with PAC1 and run on a 0.8% agarose gel to determine if recombination had occurred. Recombination was determined by the presence of bands of ˜30 kb and 3 kb indicating that recombination took place between the left arms. These plasmids were chosen to amplify by adding 50 ng of plasmid to XL10-Gold ultra-competent cells on ice for 30 minutes. The tubes were then placed in a 42° C. water bath for 30 seconds, and then placed back on ice for two minutes. Pre-warmed 42° C. NYZ+ broth was added to each tube and incubated at 37° C. for one hour in a temperature controlled shaker and then plated on LB-kanamycin plates and incubated overnight at 37° C. One colony from each transformation was selected and grown overnight in 10 mL of kanamycin-LB broth at 37° C. in a temperature controlled shaker. To isolate the plasmids, a Maxiprep was performed according to manufacturer's protocol (Qiagen) and cut with PAC1. 5 μg of plasmid DNA was transfected into AD-293 cells.

Determining Viral Titer: The cells were plated at a density of 2.2×10⁵ cells per well in 0.5 mL of DMEM growth medium in a 24-well tissue culture plate. A serial dilution of adenoviral stock was performed (10⁻² through 10⁻⁶) and each dilution was added to the appropriate well. Each sample was done in duplicate: 1. Empty virus, 2. Ad-ADAM 12S. 504, was added to each well and incubated for 48 hours at 37° C. in a 5% CO2 humidified incubator. Initial infection was determined by fluorescent microscopy 24 hours post infection.

48 hours post-infection, the media was removed via aspiration and cells were left to dry for 10 minutes under a hood. The cells were then fixed in 100% ice cold methanol and stored at −20° C. overnight. The methanol was aspirated, the cells were washed twice with 1×PBS/1% BSA, and then 0.25 mL of a 1:500 dilution of mouse anti-hexon antibody in 1×PBS/1% BSA was added to the well and incubated at 37° C. The media was removed via aspiration, washed twice with 1×PBS/1% BSA and then 0.25 mL of a 1:1000 HRP-conjugated goat anti-mouse antibody diluted in 1×PBS/1% BSA and incubated for 1 hour at 37° C. The cells were washed 2 times with 1×PBS/1% BSA and then 0.25 mL 1×DAB working substrate was added. Cells were left to incubate at room temperature until desired staining was observed then placed at 4° C. overnight to intensify staining. Finally, the DAB substrate was aspirated and 1×PBS was added.

All viral titer values were calculated using the average number of positive stained cells in 10 different field views on a 20× objective lens of a microscope and the following formula: IFU/ml=((#cells/field)×(fields/well))/(volume of diluted virus per well)×(dilution factor), per the AdEasy Viral Titer Kit protocol (Agilent Technologies).

Generation of ADAM 12S expressing Human Primary Subcutaneous Preadipocytes: In a 6-well tissue culture plate (CytoOne, Japan), human primary subcutaneous preadipocytes (ZenBio, Inc.) were grown in preadipoctye growth media (DMEM supplemented with 10% fetal bovine serum, penicillin (100 μg/mL), streptomycin (100 μg/mL), and Amphotericin B (2.5 μg/mL)). After reaching approximately 40% confluence, the cells were then infected with either 15 μl Ad-MOCK or 15 μl Ad-ADAM 12S. After 48 hours, the plate was observed for fluorescence and both bright field and fluorescent images were obtained. After 72 hours, the virus was removed by replacing the media with fresh preadipocyte growth medium.

Oil Red O staining of lipid droplets of the Ad-infected cells: Cells were grown in a 6-well tissue culture plate (CytoOne, Japan) containing preadipoctye growth media (DMEM supplemented with 10% fetal bovine serum, penicillin (100 μg/mL), streptomycin (100 μg/mL), and Amphotericin B (2.5 μg/mL)) and infected with either 15 μl Ad-MOCK or 15 μl Ad-ADAM 12S. Once lipid accumulation became noticeable under a microscope, 1.0 mL of 1×PBS was used to wash the cells. This was repeated three times before the cells were fixed with 1.0 mL of 10% formalin for 60 minutes at room temperature. Overnight the Oil Red O stain stock solution was prepared by mixing 0.3 g Oil Red O in 100 mL of isopropanol at room temperature. The stock solution was filtered the next day using Whatman #2 filter papers. When the incubation period was over, formalin was removed from the cells and they were washed with deionized water. After aspirating the water, culture wells were filled with 1.0 mL of 60% isopropanol working solution and incubated at room temperature for five minutes. The working solution of Oil Red O was prepared by diluting 30 mL of stock solution with 20 mL of distilled water. This was filtered with Whatman #1 filter papers into a Coplin jar, and immediately covered with a lid. Isopropanol working solution was aspirated from the culture wells and then 1.0 mL of Oil Red O working solution was added to each well. The 6-well plate was then incubated for five minutes at room temperature. Deionized water was used to rinse the cells until the fluid ran clear. Cells were counterstained with 1 mL of hematoxylin for one minute at room temperature. Hematoxylin was removed from the wells via aspiration. Finally, the cells were rinsed using deionized water and images were taken under a microscope (Olympus, Shinjuku, Tokyo, Japan).

RT-PCR in Primary Human Subcutaneous Preadipocytes: Total RNA was extracted from human primary subcutaneous pre-adipocytes (ZenBio) with TriReagent, DNase-digested, and quantified by Nanodrop. RT-PCR was used to analyze total RNA (1.5 μg) and determine whether hHB-EGF was present in these cells. Superscript One-step RT-PCR kit (Invitrogen) was used and each reaction was subjected to 50° C. (30 minutes), 30 cycles of 94° C. (30 seconds), 59° C. (1 minute), 72° C. (45 seconds), immediately followed by a 72° C. (15 minutes) extension process. Gel electrophoresis was used to analyze the PCR products on an 0.8 % agarose gel stained with ethidium bromide. The primer set for hHB-EGF is shown in Table 1.

TABLE 1
Primer sequences for all PCR and qPCR products. All primers are from 5′ to 3′

| Gene Name | Forward Primers | Reverse Primers |
|---|---|---|
| Human PRDM16 | GAG CAT TTC ACC CCA TCA AC | GGA GTC TGG AGC ATT TCA CC |
| Human UCP-1 | TCT ACG ACA CGG TCC AGG | GTC TGA CTT TCA CGA CCT CTG |
| Human PGC-1α | GCC AAA CCA ACA ACT TTA TCT CTT C | CAC ACT TAA GCG TTC AAT AGT C |
| Human β-actin | TCC CTG GAG AAG AGC TAC GA | AGC ACT GTG TTG GCG TAC AG |
| Human GAPDH | AAT CCC ATC ACC ATC TTC CA | TGG ACT CCA CGA CGT ACT CA |
| hHB-EGF | ATGAAGCTGCTGCCGTCGG | AGTGGGAATTAGTCATGCCC |

qPCR in Human Primary Subcutaneous Preadipocytes: qPCR was performed in a CFX connect (BioRad) according to the manufacturer's protocols using a gScript™ 1-Step SYBR Green qRT-PCR kit (Quantabio, San Diego, Calif.). Total RNA was extracted from human primary subcutaneous preadipocytes which were infected with either Ad-MOCK or Ad-ADAM 12S using a Maxwell RNA extraction kit (Promega) and following the manufacturer's protocols. Each reaction contained 138.5 ng of total RNA, and primers with a final concentration of 0.5 μM. The samples were placed in the CFX connect and the cycle program was set up to run at 50° C. for 10 minutes, 95° C. for 5 minutes, 45 cycles at 95° C. for 10 seconds followed by 60° C. for 30 seconds where the fluorescence of SYBR green was recorded. Brown fat genes, PRDM16, PGC-1α, and UCP1 were normalized to internal controls β-actin and GAPDH. Using the comparative C(T) method (Schmittgen & Livak, 2008), relative quantification was determined since the experiment was not conducted on premade Qiagen plates.

Seahorse Metabolic Analysis: Using the manufacturer's recommended protocol, metabolic data was tested for and results were gathered. Cells were plated in Seahorse XF24 assay plates at a density of 100,000 cells per well (Seahorse Bioscience, Chicopee, Mass.). The day before the assay, the sensor cartridge was hydrated overnight at 37° C. in a non-CO₂ incubator. The day of the assay, Seahorse XF Base Medium was supplemented with 1 mM pyruvate, 2 mM glutamine, and 10 mM glucose and brought to a pH of 7.4 with 0.1N NaOH. Cell growth media was removed from the assay plates after confirming that the cells had adhered at the desired cell confluence. After aspiration of cell growth medium, cells were washed with 1×PBS and 500 μL of warmed assay media was added to each well. A Seahorse XF Cell Energy Phenotype Test Kit (Seahorse Bioscience) was utilized to observe metabolic rate. Stock solutions of FCCP (100 μM) and Oligomycin (100 μM) were prepared according to the manufacturer's protocol. The stressor mix was made with 2400 μL of assay medium, and 300 μL each of Oligomycin and FCCP stock solutions. Catecholamines working solution (4 μg/mL) was made by diluting 6 μL of sock catecholamines (1 mg/mL) with 1.5 mL of assay medium. The assay was run according to manufacturer's recommendations for 4 cycles of 3 loops each time: first basal, then catecholamines, and finally the stressor mix. Each loop injected the drug of choice: catecholamines (50 μl), or the stressor mix (56 μl) before shaking for 1 minute and then probing for 3 minutes.

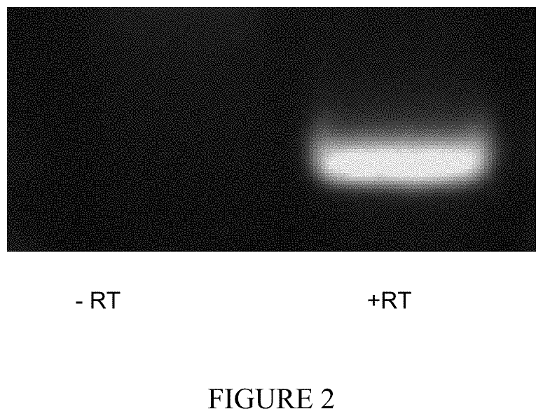

RT-PCR in Human Primary Preadipocytes: Total RNA was isolated from primary human preadipocytes obtained from ZenBio to confirm the expression of native HB-EGF. Using human HB-EGF primers, a 627 bp HB-EGF PCR product resulted in the presence of reverse transcriptase (+RT) (FIG. 2). This suggests endogenous HB-EGF is found in human primary preadipocytes. Acting as a negative control, no detectable PCR product was found in the absence of reverse transcriptase (−RT).

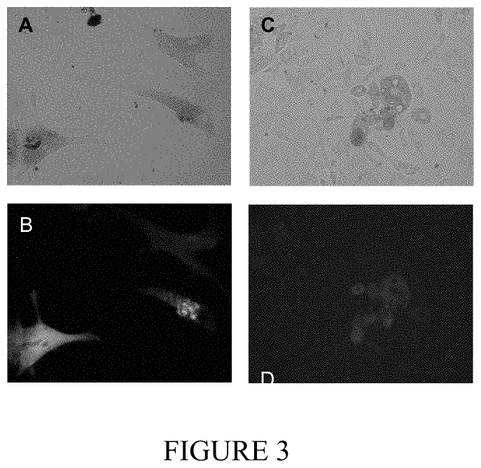

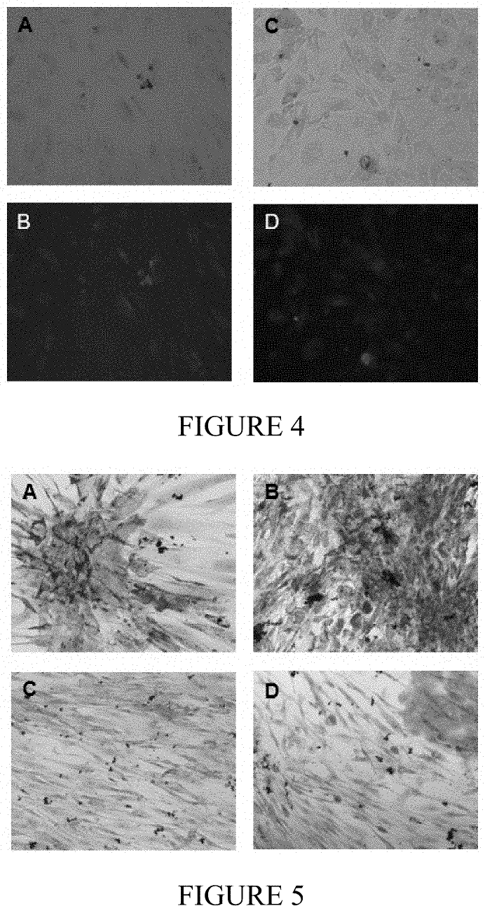

Lipid Accumulation and Oil Red O Staining in Infected Cells: Human primary preadipocytes were infected with either Ad-ADAM 12S or Ad-MOCK. Fluorescent microscopy 48 hours after infection confirmed the infection of the cells by the adenovirus due to the detection of GFP in both ADAM 12S and MOCK infected cells (FIGS. 3 and 4). Lipid droplet accumulation became noticeable in the Ad-ADAM 12S infected cells three weeks after infection (FIG. 3). Ad-MOCK infected cells did not demonstrate noticeable lipid accumulation (FIG. 4). To confirm that the observed accumulation was in fact lipids, Oil Red O staining was conducted. The cells were stained with Oil Red O and counterstained with hematoxylin. The Ad-ADAM 12S infected cells had a tremendous amount of Oil Red O staining (FIG. 5A,B) while the Ad-MOCK infected cells did not (FIG. 5 C,D). Staining is typical of brown adipose tissue.

Differential Gene Expression in Human Primary Preadipocytes: Three weeks after infection, total RNA was isolated from Ad-ADAM 12S and Ad-MOCK infected human primary preadipocytes. qPCR was conducted to analyze for the expression of BAT marker genes PRDM 16, UCP-1, and PGC-1α. Each gene had three replicates and the RNA was isolated from a single well of a six well plate. Ad-ADAM 12S infected cells showed statistically significant upregulation of PGC-1α and statistically significant downregulation of PRDM 16 (Table 2). UCP-1 was upregulated but not statistically significant (Table 2). BAT marker genes were normalized to β-actin and GAPDH.

| Gene name | Fold Change | p-value |
|---|---|---|
| PRDM16 | −2407 | 0.0008 |
| UCP-1 | 3.47 | 0.2294 |
| PGC-1α | 21.4 | 0.0018 |

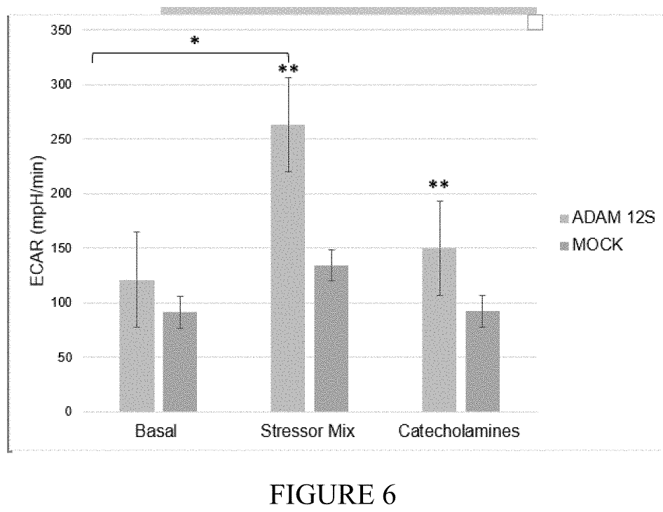

Seahorse Metabolic Assay: Human Primary Preadipocytes infected with either Ad-ADAM 12S or Ad-MOCK were analyzed to determine the oxidative phosphorylation and glycolytic profiles. The extracellular acidification rate (ECAR) is a measure of the rate of glycolysis of the cells. ECAR was significantly increased in ADAM 12S infected cells when stressed with the stressor mix (FCCP+Oligomycin) compared to their basal rate (FIG. 6). When comparing ADAM 12S infected cells to MOCK infected cells, there is no significant difference between their basal rates. After being stressed with catecholamines, ADAM 12S cells have a significantly higher ECAR compared to MOCK infected cells (FIG. 6). When the stressor mix of FCCP+Oligomycin is injected, the ADAM 12S infected cells show a significant increase in ECAR compared to the MOCK infected cells (FIG. 6). The glycolytic rate of ADAM 12S infected cells is significantly increased compared to MOCK infected cells.

All combinations of method or process steps as used herein can be performed in any order, unless otherwise specified or clearly implied to the contrary by the context in which the referenced combination is made.

All ranges and parameters, including but not limited to percentages, parts, and ratios, disclosed herein are understood to encompass any and all sub-ranges assumed and subsumed therein, and every number between the endpoints. For example, a stated range of “1 to 10” should be considered to include any and all subranges between (and inclusive of) the minimum value of 1 and the maximum value of 10; that is, all subranges beginning with a minimum value of 1 or more (e.g., 1 to 6.1), and ending with a maximum value of 10 or less (e.g., 2.3 to 9.4, 3 to 8, 4 to 7), and finally to each number 1, 2, 3, 4, 5, 6, 7, 8, 9, and 10 contained within the range.

All references to singular characteristics or limitations of the present disclosure shall include the corresponding plural characteristic or limitation, and vice versa, unless otherwise specified or clearly implied to the contrary by the context in which the reference is made.

The compositions, and corresponding methods of the present disclosure can comprise, consist of, or consist essentially of the essential elements and limitations of the disclosure as described herein, as well as any additional or optional ingredients, components, or limitations described herein or otherwise useful in the general inventive concepts.

The compositions of the present disclosure may also be substantially free of any optional or selected component or feature described herein, provided that the remaining composition still contains all of the required elements or features as described herein. In this context, and unless otherwise specified, the term “substantially free” means that the selected composition contains less than a functional amount of the optional component, typically less than 0.1% by weight, and also including zero percent by weight of such optional or selected component.

To the extent that the terms “include,” “includes,” or “including” are used in the specification or the claims, they are intended to be inclusive in a manner similar to the term “comprising” as that term is interpreted when employed as a transitional word in a claim. Furthermore, to the extent that the term “or” is employed (e.g., A or B), it is intended to mean “A or B or both A and B.” When the Applicant intends to indicate “only A or B but not both,” then the term “only A or B but not both” will be employed. Thus, use of the term “or” herein is the inclusive, and not the exclusive use. In the present disclosure, the words “a” or “an” are to be taken to include both the singular and the plural. Conversely, any reference to plural items shall, where appropriate, include the singular.

In some aspects, it may be possible to utilize the various inventive concepts in combination with one another. Additionally, any particular element recited as relating to a particularly disclosed embodiment should be interpreted as available for use with all disclosed embodiments, unless incorporation of the particular element would be contradictory to the express terms of the embodiment. Additional advantages and modifications will be readily apparent to those skilled in the art. Therefore, the disclosure, in its broader aspects, is not limited to the specific details presented therein, the representative apparatus, or the illustrative examples shown and described. Accordingly, departures may be made from such details without departing from the spirit or scope of the general inventive concepts.

While the invention has been illustrated and described in detail in the drawings and foregoing description, the same is to be considered as illustrative and not restrictive in character. It should be understood that only the exemplary embodiments have been shown and described and that all changes and modifications that come within the spirit of the invention are desired to be protected.

1. A method of enhancing at least one BAT-like expression level in an individual, the method comprising
identifying an individual in need of increased expression of at least one BAT-like marker;
providing an expression vector comprising a nucleotide sequence encoding ADAM 12S operatively linked to a promoter;
contacting at least one cell of the individual with the expression vector.

2. The method of claim 1, were in the individual has symptoms of at least one of obesity, metabolic syndrome, type II diabetes, pre-diabetes.

3. The method of claim 1, wherein the expression vector is a plasmid.

4. The method of claim 1, wherein the expression vector is a viral vector.

5. The method of claim 1, wherein the expression vector is an adenoviral vector.

6. The method of claim 1, wherein the vector does not comprise Ad-HB-EGF.

7. The method of claim 1, wherein the cell is a human primary adipocyte.

8. The method of claim 1, wherein contracting the cell results in at least one of the following:
lipid droplet accumulation,
increase in the number of mitochondria,
cellular quiescence,
increased BAT gene expression (PRDM16, PGC-1a, UCP-1),
decreased WAT gene expression (PPARg, C/EBPa, AKT-1),
increased glycolysis,
increased oxygen consumption, and
increased extracellular acidification rates compared to control cells.

9. A composition for altering at least one BAT expression level in a cell, the composition comprising an expression vector comprising a nucleotide sequence encoding ADAM 12S operatively linked to a promoter, wherein the composition does not comprise Ad-HB-EGF.

10. The composition of claim 9, wherein the expression vector is a plasmid.

11. The composition of claim 9, wherein the expression vector is a viral vector.

12. The composition of claim 9, wherein the expression vector is an adenoviral vector.

13. The composition of claim 9, wherein the cell is differentiated mammalian cell.

14. The composition of claim 9, wherein the cell is a human primary adipocyte.

15. A method of treating a condition associated with at least one of obesity, metabolic syndrome, and insulin resistance by reprogramming at least one differentiated cell in an individual, the method comprising
identifying an individual diagnosed with at least one of obesity, metabolic syndrome, and insulin resistance;
providing an expression vector comprising a nucleotide sequence encoding ADAM 12S operatively linked to a promoter;
contacting at least one cell of the individual with the expression vector.

16. The method of claim 15, wherein the expression vector is a plasmid.

17. The method of claim 15, wherein the expression vector is an adenoviral vector.

18. The method of claim 15, wherein the vector does not comprise Ad-HB-EGF.

19. The method of claim 15, wherein the cell is a human primary adipocyte.

20. The method of claim 15, wherein contracting the cell results in at least one of the following:
lipid droplet accumulation,
increase in the number of mitochondria,
cellular quiescence,
increased BAT gene expression (PRDM16, PGC-1a, UCP-1),
decreased WAT gene expression (PPARg, C/EBPa, AKT-1),
increased glycolysis,
increased oxygen consumption, and
increased extracellular acidification rates compared to control cells.
